(19) United States
(12) Patent Application Publication
(10) Pub. No.: US 2005/0251928 A1
(43) Pub. Date: Nov. 17, 2005

(54) Method for stripping artificial color from keratin fibers

(75) Inventors: Sylvain Kravtchenko, Asnieres (FR); Aude Livoreil, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/129,331

(22) Filed: May 16, 2005

Related U.S. Application Data
(60) Provisional application No. 60/580,385, filed on Jun. 18, 2004.

(30) Foreign Application Priority Data
May 17, 2004 (FR) 0405353

(51) Int. Cl. A61K 7/13

(52) U.S. Cl. 8/405

(57) ABSTRACT

The present invention relates to stripping artificial color from keratin fibers with a composition containing at least one phosphine and/or an acid-addition salt thereof.

### REFERENCE TO PRIOR APPLICATIONS

This application claims priority to U.S. provisional application 60/580,385 filed Jun. 18, 2004, and to French patent application 0405353 filed May 17, 2004, both incorporated herein by reference.

### FIELD OF THE INVENTION

The present invention relates to the use, for stripping artificial color from keratin fibers, and in particular from human keratin fibers such as hair, of a composition comprising at least one phosphine and/or an acid-addition salt thereof. This composition is sometimes referred to as a stripping composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is known practice to dye keratin fibers, and in particular human keratin fibers such as the hair, with dye compositions containing oxidation dyes and/or direct dyes.

The dyeing performed with oxidation dyes, or “oxidation dyeing”, is permanent dyeing; it comprises as oxidation dyes oxidation dye precursors and couplers.

Oxidation dye precursors, which are commonly known as “oxidation bases”, are compounds that are initially colorless or weakly colored, which develop their dyeing power on the hair in the presence of oxidizing agents added at the time of use, leading to the formation of colored compounds and dyes. The formation of these colored compounds and dyes results either from an oxidizing condensation of the “oxidation bases” with themselves, or from an oxidizing condensation of the “oxidation bases” with coloration modifiers, which are commonly known as “couplers” and which are generally present in the dye compositions used in oxidation dyeing.

The variety of molecules used as oxidation bases and couplers allows a wide range of colors to be obtained.

The dyeing performed with direct dyes gives a semi-permanent or temporary coloration; direct dyes give the natural color of the hair a more or less pronounced color change that may be resistant to shampooing several times.

The direct dyes conventionally used are chosen especially from nitrobenzene direct dyes, azo direct dyes, quinone and in particular anthraquinone direct dyes, azine direct dyes, triarylmethane direct dyes, indoamine direct dyes and natural direct dyes.

Oxidation dyes in combination with direct dyes may be used to vary the shades obtained by oxidation dyeing, or to enrich them with glints.

For various reasons, such as the wish to partially or totally modify the shade thus given to a head of hair by an oxidation dyeing or direct dyeing operation, or the wish to remove this coloration, it may be desired to partially or totally destroy the dyes thus formed in or on the hair. Stripping of the artificial color from the keratin fibers is then performed.

This stripping is generally performed via processes using oxidizing or reducing systems.

In the oxidizing systems, the oxidizing agents conventionally used are hydrogen peroxide or compounds capable of producing peroxygenated compounds by hydrolysis.

Among the reducing systems, it is known practice, from German patent DE 1 151 242, to use hydroxymethanesulfinic acid at a pH of between 7 and 9, to bleach dyed hair. The use of sodium sulfite (Na₂SO₃) is also disclosed in U.S. Pat. No. 2,149,319 and U.S. Pat. No. 3,838,966 and patent application JP-04-356 413A.

It is also known practice to strip keratin fibers using reducing agents at acidic pH. Thus, it is known practice to use sodium hydroxymethanesulfinate as reducing agent for dyed hair, which is mixed with an acidic aqueous solution at the time of use. Patent application EP 0 943 316 discloses the use of a combination at acidic pH comprising ascorbic acid and α-oxocarboxylic acid to strip the hair.

A process for stripping the artificial color from keratin fibers is also described in U.S. Pat. No. 3,892,845, and consists in applying to the fibers an aqueous composition comprising a combination of two types of reducing agent, a reducing agent for the dye and a reducing agent for the disulfide covalent bonds of keratin; the dye-reducing agent is a zinc, potassium, sodium or calcium hydroxymethanesulfinate or hydrosulfite, and the keratin-reducing agent is especially thioglycolic acid, a potassium or sodium bisulfate or bisulfite, potassium disulfide, thiourea or certain phosphorus compounds, such as phosphines.

However, none of these prior art techniques gives rise to sufficiently efficient stripping of keratin fibers, in particular for fundamental shades and shades with golden and ash glints.

### SUMMARY OF THE INVENTION

The present invention provides compositions for stripping artificial color from keratin fibers which do not have the drawbacks of known stripping products of the prior art, and in particular provides more efficient compositions for stripping artificial color from keratin fibers.

### DETAILED DESCRIPTION OF THE PREFERRED EMBODIMENTS

One subject of the present invention is a composition useful for stripping artificial color from keratin fibers, comprising, preferably in a cosmetically acceptable medium, at least one phosphine and/or acid-addition salt thereof (phosphine/salt). This phosphine/salt preferably is present as major reducing agent. In preferred embodiments this invention composition is in contact with keratin fibers to be stripped.

Another subject of the present invention is a process for stripping artificial color from keratin fibers using a composition according to the invention.

The present invention also relates to a multi-compartment device for dyeing keratin fibers and then stripping the artificial color from the keratin fibers.

The present invention allows artificial color to be stripped from keratin fibers dyed with a wide range of oxidation dyes and/or direct dyes, which do not induce lightening of the natural base of the keratin fibers, which are more practical to use, for example which do not require mixing at the time of use, and which limit the sensitization of the keratin fibers.

In the context of the present invention, the term “major reducing agent” means that when the composition contains additional reducing agents other than the invention phosphine/salt, they are always present in a weight amount less than the weight amount of the phosphine/salt.

In the definitions hereinbelow, the hydrocarbon-based radicals are linear or branched, cyclic or acyclic, saturated or unsaturated, and contain, unless otherwise mentioned, from 1 to 30 carbon atoms and preferably from 1 to 10 carbon atoms.

The term “unsaturated hydrocarbon-based radical” means a hydrocarbon-based radical containing from 2 to 30 carbon atoms and preferably from 2 to 10 carbon atoms, and comprising one or more double and/or triple bonds.

As examples of phosphines that are useful in the context of the present invention, mention may be made of tri(hydroxymethyl)phosphine; tri(hydroxy-propyl)phosphine; bis(hydroxymethyl)(phenyl)phosphine; allyldiphenylphosphine; benzyloxy(diisopropylamino)methylphosphine; 2,2′-bis(dicyclohexylphosphino)-1,1′-binaphthyl; 2,2′-bis[bis(3,5-dimethylphenylphosphino)]-1,1′-binaphthyl; 1,4-bis[bis(3,5-dimethylphenyl)phosphino]butane; 1,2-bis[bis(3,5-dimethylphenyl)phosphino]ethane; bis[bis(3,5-dimethylphenyl)phosphino]-methane; 1,5-bis[bis(3,5-dimethylphenyl)phosphino]pentane; 1,3-bis[bis(3,5-dimethylphenyl)phosphino]propane; 2,2′-bis[bis(3,5-ditrifluoromethylphenyl)phosphino]-1,1′-binaphthyl; 1,4-bis[bis(3,5-ditrifluoromethylphenyl)phosphino]butane; 1,2-bis[bis(3,5-ditrifluoromethylphenyl)phosphino]ethane; bis[bis(3,5-ditrifluoromethylphenyl)phosphino]methane; 1,5-bis-[bis(3,5-ditrifluoromethylphenyl)phosphino]pentane; 1,3-bis[bis(3,5-ditrifluoromethylphenyl)phosphino]-propane; bis(3,5-di-tert-butylphenyl)chlorophosphine; bis(3,5-di-tert-butylphenyl)phosphine; 1,2-bis(di-tert-butylphosphino)benzene; 1,4-bis(di-tert-butylphosphino)butane; 1,2-bis(di-tert-butylphosphino)ethane; 1,1′-bis(di-tert-butylphosphino)ferrocene; 1,3-bis(di-tert-butylphosphinomethyl)benzene; 1,3-bis(di-tert-butylphosphino)propane; 1,2-bis(dichlorophosphino)benzene; 1,3-bis(dichlorophosphino)benzene; 1,4-bis-(dichlorophosphino)benzene; 1,4-bis(dichlorophosphino)butane; 1,2-bis(dichlorophosphino)-1,2-dimethyl-hydrazine; 1,2-bis(dichlorophosphino)ethane; 1,1′-bis(dichlorophosphino)ferrocene; bis(dichlorophosphino)methane; 1,3-bis(dichlorophosphino)propane; 1,2-bis(dicyclohexylphosphino)benzene; 2,2′-bis(dicyclohexylphosphino)-1,1′-binaphthyl; 1,4-bis(dicyclohexylphosphino)butane; (2R,3R)bis(dicyclohexylphosphino)butane; (2S,3S)bis(dicyclohexylphosphino)butane; 1,2-bis(dicyclohexylphosphino)ethane; bis(dicyclohexylphosphino)methane; 1,3-bis(dicyclohexylphosphino)propane; bis(diethylamino)chlorophosphine; bis[2-(4-diethylaminomethylphenyl)phenylphosphino]ethyl ether; 1,2-bis(diethylphosphino)ethane; bis(diisopropylamino)chlorophosphine; 1,1′-bis(diisopropylphosphino)ferrocene; bis(3,5-dimethyl-4-methoxyphenyl)chlorophosphine; bis(3,5-dimethyl-4-methoxyphenyl)phosphine; bis(3,5-dimethylphenyl)chlorophosphine; bis(3,5-dimethylphenyl)diethylaminophosphine; bis(3,5-dimethylphenyl)phosphine; 1,2-bis(dimethylphosphino)benzene; 1,4-bis(dimethylphosphino)butane; 1,2-bis(dimethylphosphino)ethane; bis(dimethylphosphino)methane; 1,3-bis-(dimethylphosphino)propane; 1,2-bis(diphenylphosphino)benzene; 1,3-bis(diphenylphosphino)benzene; 1,4-bis-(diphenylphosphino)benzene; 2,2′-bis(diphenylphosphino)-1,1′-binaphthyl; 1,4-bis(diphenylphosphino)butane; 1,2-bis(diphenylphosphino)ethane; cis-1,2-bis(diphenylphosphino)ethylene; trans-1,2-bis-(diphenylphosphino)ethylene; bis(2-diphenylphosphino)ethyl ether; bis(2-diphenylphosphinoethyl)phenylphosphine; 1,1′-bis(diphenylphosphino)ferrocene; 1,1′-bis(diphenylphosphino)ferrocenenickel(II) dichloride; 1,6-bis(diphenylphosphino)hexane; bis(diphenylphosphino)methane; 1,5-bis(diphenylphosphino)pentane; 1,3-bis(diphenylphosphino)propane; bis(3,5-ditrifluoromethyl)chlorophosphine; bis(3,5-ditrifluoromethylphenyl)phosphine; 1,2-bis(ditrifluoromethylphosphino)ethane; bis(2-furyl)chlorophosphine; bis(2-furyl)phosphine; bis(hydroxymethyl)phenylphosphine; bis(4-methoxyphenyl)phenylphosphine; 1,2-bis[(methoxyphenyl)phenylphosphino]ethane; bis(4-methylphenyl)chlorophosphine; bis(4-methylphenyl)phosphine; 1,2-bis-(phenylphosphino)ethane; 1,3-bis(phenylphosphino)propane; bis-2-[(phenyl)(3-pyridyl)phosphinoethyl) ether; 1,2-bis(phosphino)benzene; 1,2-bis(phosphino)ethane; 1,1′-bis(phosphino)ferrocene; bis(phosphino)methane; bis(pyrrolidino)methylphosphine; bis(4-tri-fluoromethylphenyl)phosphine; 1,2-bis[(trifluoromethyl)phosphino]ethane; bis(trimethylphosphine)cobalt dichloride; bis(trimethylphosphine)copper dichloride; bis(trimethylphosphine)iron dichloride; bis(trimethylphosphine)zinc dichloride; tert-butylbis(trimethylsilyl)phosphine; butyldichlorophosphine; tert-butyl-dichlorophosphine; tert-butyldiethylphosphine; butyldiphenylphosphine; tert-butyldiphenylphosphine; chlorodiisopropylphosphine; 2-chloroethyldiphenylphosphine; cyclohexyldichlorophosphine; cyclohexyl-(diethylamino)chlorophosphine; cyclohexyl(dimethylamino)chlorophosphine; cyclohexyldiphenylphosphine; diallylphenylphosphine; dibenzylphosphine; di-tert-butylchlorophosphine; di-tert-butylchlorophosphine-borane complexes; dibutylphenylphosphine; dibutylphosphine; di-tert-butylphenylphosphine; di-tert-butylphosphine; di-tert-butylphosphine-borane complexes; 2-(di-tert-butylphosphino)biphenyl; dicyclohexylchlorophosphine; dicyclohexylphosphine; dicyclohexylphosphine-borane complexes; 2-(dicyclohexylphosphino)biphenyl; 2-dicyclo-hexylphosphino-2′-(N,N-dimethylamino)biphenyl; diethyl-aminodiethylphosphine; diethylchlorophosphine; diethyl-phenylphosphine; diethylphosphine; diisobutylphosphine; dimethylaminodichlorophosphine; (4-dimethylaminophenyl)diphenylphosphine; dimethylchlorophosphine; dimethylphenylphosphine; dimethyl(trimethylsilyl)phosphine; diphenylphosphine; diphenylphosphinic acid; N-[(diphenylphosphinyl)methyl]-N-methylaniline; diphenylchlorophosphine; diphenylphosphine; o-diphenyl-phosphinobenzoic acid; diphenylpropylphosphine; diphenyl(p-tolyl)phosphine; diphenyl(trimethylsilyl)phosphine; diphenylvinylphosphine; divinylphenylphosphine; ethyldichlorophosphine; ethyldiphenylphosphine; isopropyldichlorophosphine; 2-methoxy(dichlorophosphino)benzene; methoxydiethoxyphosphine; 4-methoxy-phenyl(diethylamino)chlorophosphine; 4-methoxyphenyl-(dimethylamino)chlorophosphine; (2-methoxyphenyl)methylphenylphosphine; 2-methoxyphosphinobenzene; methyl-2,3-bis-O-diphenylphosphino-4,6-O-benzylidene-glucopyranoside; methyldichlorophosphine; methyl-diphenylphosphine; (5-methyl-2-isopropylcyclohexyl)diphenylphosphine; methylphenylchlorophosphine; phenylphosphine; 9H-9-phosphabicyclo[3,3,1]nonane (phobane); 9H-9-phosphabicyclo[4,2,1]nonane (phobane); propyldichlorophosphine; tetraphenylbiphosphine; tri-tert-butylphosphine; tricyclohexylphosphine; tricyclopentylphosphine; triethylphosphine; triisobutylphosphine; triisopropylphosphine; trimethylphosphine; tri-n-octylphosphine; triphenylphosphine dibromide; triphenylphosphine dichloride; triphenylphosphine hydrobromide; tripropylphosphine; tris[3,5-bis(trifluoromethyl)phenyl]phosphine; tris(2-carboxyethyl)phosphine hydrochloride; tris(3-chlorophenyl)phosphine; tris(4-chlorophenyl)phosphine; tris(2-cyanoethyl)phosphine; tris(2,6-dimethoxyphenyl)phosphine; tris(3-fluorophenyl)phosphine; tris(4-fluorophenyl)phosphine; tris(2-furyl)phosphine; tris(hydroxymethyl)phosphine; tris(2-methoxyphenyl)phosphine; tris(3-methoxyphenyl)phosphine; tris(4-methoxyphenyl)phosphine; tris(3-methoxypropyl)phosphine; tris(4-morpholino)phosphine; tris(1-naphthyl)phosphine; tris(2-thienyl)phosphine; tris(2,4,6-trimethylphenyl)phosphine; tris(trimethylsilyl)phosphine; tri(m-tolyl)phosphine; tri(o-tolyl)phosphine; tri(p-tolyl)phosphine; 2-cyanoethyldiphenylphosphine; 2-dicyclohexylphosphino-2]-methylbiphenyl; 2-methoxyphosphinobenzene; benzyldiphenylphosphine; bis(2,4,6-trimethylphenyl)phosphine; bis(2-cyanoethyl)phosphine; bis(3,4,5-trimethoxyphenyl)chlorophosphine; bis(3,4,5-trimethoxyphenyl)phosphine; bis(4-fluorophenyl)chlorophosphine; bis(4-methoxyphenyl)chlorophosphine; bis(4-trifluoromethylphenyl)chlorophosphine; bis(diethylamino)methylphosphine; bis(diethylamino)phenylphosphine; bis(di-tert-butylphosphino)pentane; bis(o-tolyl)chlorophosphine; bis(o-tolyl)phosphine; dicyclohexylphenylphosphine; diisopropylphosphine; di-tert-butylhydroxyphosphine; di-tert-butylmethylphosphine; methyldichlorophosphine; di-tert-butylmethylphosphine tetrafluoroborate; eicosyl phobane; methylphosphine-borane complexes, phenylphosphineborane complexes; tert-butylbiphenylphosphine; tert-butylphosphine; triisopropylphosphine; tri-n-butylphosphine; triphenylphosphine-borane; tri(3,5-dimethyl-4-methoxy)phosphine, and acid-addition salts thereof.

According to one particular embodiment of the invention, the phosphine(s) is (are) chosen from the compounds of formula (I) below: 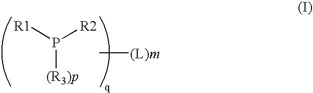
in which:
L is a linker that represents a covalent bond or a divalent hydrocarbon-based radical optionally comprising one or more hetero atoms chosen from an oxygen atom, a sulfur atom, a nitrogen atom and a silicon atom;
m is an integer equal to 0 or 1;
q is an integer equal to 1 or 2;
p is an integer equal to 0 or 1;
R₁, R₂ and R₃, which may be identical or different, represent:
   a hydrogen atom;
   a halogen atom;
   a hydroxyl radical;
   a carboxyl radical;
   a monovalent hydrocarbon-based radical optionally comprising one or more hetero atoms chosen from a sulfur atom, an oxygen atom, a nitrogen atom, a phosphorus atom and a silicon atom, optionally substituted with one or more radicals chosen from:
      a halogen atom,
      a hydroxyl radical,
      an alkoxy radical,
      a haloalkyl radical,
      an amino radical,
      a carboxyl radical,
      an alkoxycarbonyl radical,
      an amido radical,
      an alkylaminocarbonyl radical,
      an acylamino radical,
      a mono- or di(alkyl)amino radical,
      a mono- or di(hydroxyalkyl)amino radical,
      an N-aryl-N-alkylamino radical,
      an aromatic or heteroaromatic ring, which is unsubstituted or substituted with one or more radicals chosen from a halogen atom, a hydroxyl radical, an alkoxy radical and a mono- or di(alkyl)amino radical,
      a cyano radical,
      a radical that increases the solubility of the phosphine in water, such as sulfonate, sulfinate, phosphonate or carboxylate radicals,
      a substituted or unsubstituted, aromatic or non-aromatic heterocyclic radical;
   a substituted or unsubstituted aryl radical;
   a substituted or unsubstituted arylalkyl radical;
   an arylalkyloxy radical;
   a substituted or unsubstituted, aromatic or non-aromatic heterocyclic radical;
   a silyl radical;
      it being understood that:
when q=1, m=0 and p=1;
when q=2, m=1 and p=0 or 1, with:
   when p=0, the linker L is attached to the phosphorus atom;
   when p=1, the linker L is attached to one of the radicals R₁, R₂ and R₃, and acid-addition salts thereof.

In all the above meanings, when a radical is substituted, the substituents are chosen from halo, hydroxyl, alkyl, haloalkyl, alkoxy, amino, mono- or dialkylamino, mono- or dihydroxyalkylamino and carboxyl. For example, the radical p-methoxyphenyl is a substituted aryl radical.

Preferably, the radicals R₁, R₂ and R₃ do not simultaneously represent a hydrogen atom.

Advantageously, but optionally, at least one of the radicals R₁, R₂ and R₃ denotes, as hydrocarbon-based radical, an optionally substituted alkyl radical.

According to one particular embodiment of the invention, R₁, R₂ and R₃ are chosen from a hydrogen atom; an alkyl radical; a cycloalkyl radical optionally substituted with one or more alkyl radicals; an alkoxy radical; an alkoxyalkyl radical; a haloalkyl radical; a cyanoalkyl radical; a hydroxyalkyl radical; a carboxyalkyl radical; a halogen atom; a hydroxyl radical; a carboxyl radical; an alkenyl radical; a mono- or dialkylamino radical; an N-aryl-N-alkylaminoalkyl radical; an aryl radical optionally substituted with one or more radicals chosen from an alkyl radical, an alkoxy radical, a mono- or dialkylamino radical, a mono- or dialkylaminoalkyl radical, a haloalkyl radical, a hydroxyl radical, a carboxyl radical, a halogen atom, an aryl radical substituted with a mono- or dialkylaminoalkyl radical; an arylalkyl radical; an arylalkyloxy radical; a pyrrolidino radical; a furyl radical; a morpholino radical; a thienyl radical; a pyridyl radical; a trialkylsilyl radical; an alkyl radical substituted with a pyrrolidino radical, a furyl radical, a morpholino radical or a thienyl radical.

By way of example, R₁, R₂ and R₃ may be chosen from a hydrogen atom; a methyl radical; an ethyl radical; a propyl radical; an isopropyl radical; an n-butyl radical; an isobutyl radical; a tert-butyl radical; an octyl radical; a cyclohexyl radical; a cyclopentyl radical; a methoxy radical; an ethoxy radical; a methoxypropyl radical; a chloroethyl radical; a cyanoethyl radical; a hydroxymethyl radical; a hydroxypropyl radical; a carboxyethyl radical; a chlorine atom; a hydroxyl radical; a carboxyl radical; a trifluoromethyl radical; a chloromethyl radical; an allyl radical; a vinyl radical; a dimethylamino radical; a diethylamino radical; a di(isopropyl)amino radical; a phenyl radical; an o-tolyl radical; an m-tolyl radical; a p-tolyl radical; a dimethylphenyl radical; a trimethylphenyl radical; an o-methoxyphenyl radical; an m-methoxyphenyl radical; a p-methoxyphenyl radical; a dimethoxyphenyl radical; a trimethoxyphenyl radical; an o-(dimethylamino)phenyl radical; an m-(dimethylamino)phenyl radical; a p-(dimethylamino)phenyl radical; a di(tert-butyl)phenyl radical; a tri(tert-butyl)phenyl radical; a trifluoromethylphenyl radical; a bis(trifluoromethyl)phenyl radical; an o-fluorophenyl radical; an m-fluorophenyl radical; a p-fluorophenyl radical; an o-chlorophenyl radical; an m-chlorophenyl radical; a p-chlorophenyl radical; an o-hydroxyphenyl radical; an m-hydroxyphenyl radical; a p-hydroxyphenyl radical; a 4-(diethylaminomethyl)phenyl radical; a 3,5-dimethyl-4-methoxyphenyl radical; a 2-methylbiphenyl radical; a benzyl radical; a benzyloxy radical; a naphthyl radical; a morpholino radical; a morpholinomethyl radical; a pyrrolidino radical; a furyl radical; a pyridyl radical; a thienyl radical; a trimethylsilyl radical; a 2-(4-diethylaminomethyl-phenyl)phenyl radical; a 5-methyl-2-isopropylcyclohexyl radical; an N-methyl-N-phenylaminomethyl radical; a carboxyphenyl radical.

The phosphines that are useful in the context of the invention may be optionally salified with strong mineral acids, for instance HCl, HBr, H₂SO₄ or HBF₄ or organic acids, for instance acetic acid, lactic acid, tartaric acid, citric acid or succinic acid.

According to one particular embodiment of the invention, the phosphine(s) that is (are) useful in the context of the invention is (are) chosen from monophosphines. For example, when the phosphine(s) is (are) of formula (I), q is then preferably equal to 1.

Examples of monophosphines that may be mentioned include those listed previously.

Preferably, the monophosphines are chosen from trihydroxymethylphosphine; trihydroxypropylphosphine; bis(hydroxymethyl)phenylphosphine.

According to another particular embodiment of the invention, the phosphine(s) that is (are) useful in the context of the invention is (are) diphosphines. When the phosphine(s) is (are) of formula (I), q is then preferably equal to 2.

Preferably, p is equal to 0 and the linker L is a covalent bond or a divalent radical chosen from a binaphthylene radical; a methylene radical; an ethylene radical; a propylene radical; a butylene radical; a pentylene radical; a hexylene radical; a phenylene radical; a meta-dimethylenebenzene radical; an N-methyl-N′-methylhydrazo radical; a vinylene radical; a diethyleneoxy radical.

As examples of diphosphines that are useful in the context of the invention, mention may be made of those listed previously.

According to one particular embodiment of the invention, the phosphine(s) that is (are) useful in the context of the invention is (are) soluble in a cosmetically acceptable medium. Preferably, the phosphine(s) that is (are) useful in the context of the invention is (are) water-soluble.

In the context of the present invention, the term “water-soluble” means any phosphine whose solubility in water is greater than 0.01% at 20° C.

The stripping composition of the invention may also contain additional reducing agents other than phosphines, such as those described in patent application EP 0 943 316. Mention may be made especially of sulfinates, sugars, reductones and α-oxocarboxylic acids such as oxalic acid, glyoxalic acid, pyruvic acid or α-ketoglutaric acid. When they are present, these additional reducing agents are usually, and preferably always, present in a weight amount less than the weight amount of the phosphines.

According to one particular embodiment of the invention, the stripping composition comprises as sole reducing agent the at least one phosphine/salt as defined previously.

The phosphine(s) and salts thereof that is (are) useful in the context of the invention is (are) generally present in an amount of between 0.001% and 30% by weight approximately, preferably between 3% and 25% by weight and even more preferably between 6% and 20% by weight relative to the total weight of the stripping composition.

The stripping composition that is useful in the invention may also contain various adjuvants such as those conventionally used in stripping compositions, such as anionic, cationic, nonionic, amphoteric or zwitterionic surfactants or mixtures thereof, mineral or organic thickeners, and in particular anionic, cationic, nonionic and amphoteric polymeric associative thickeners, nonionic, anionic, amphoteric, zwitterionic or cationic conditioning polymers or mixtures thereof, preferably cationic or amphoteric substantive polymers, penetrating agents, sequestering agents, fragrances, dispersants, volatile or non-volatile, modified or unmodified silicones, film-forming agents, ceramides, preserving agents, opacifiers, mineral or plant oils, waxes and vitamins.

Needless to say, a person skilled in the art will take care to select this or these optional additional compound(s) such that the advantageous properties intrinsically associated with the stripping composition that is useful in the invention preferably are not, or are not substantially, adversely affected by the envisaged addition(s).

The cosmetically acceptable medium generally contains water or a mixture of water and of at least one organic solvent to dissolve the reducing agents and/or adjuvants that would not be sufficiently water-soluble. Examples of organic solvents that may be mentioned include C₁-C₄ alkanols, such as ethanol and isopropanol; glycerol; glycols and glycol ethers, for instance 2-butoxyethanol, propylene glycol, dipropylene glycol, hexylene glycol, propylene glycol monomethyl ether, diethylene glycol monoethyl ether and monomethyl ether, and also aromatic alcohols, for instance benzyl alcohol or phenoxyethanol, similar products and mixtures thereof.

The solvents may then be present in proportions for example preferably between 0.5% and 20% and more particularly from 2% to 10% by weight relative to the total weight of the stripping composition.

The pH of the stripping composition that is useful in the invention is not limited but is preferably between 2 and 12. Depending on the phosphine, a person skilled in the art will adjust the optimum pH of the stripping composition, i.e. the pH value for which the stripping obtained is optimal.

According to one particular embodiment of the invention, the phosphine is trihydroxymethylphosphine and the pH of the stripping composition is about 3.

According to another particular embodiment of the invention, the phosphine is trihydroxypropylphosphine and the pH of the stripping composition is about 9.

The pH of the stripping composition that is useful in the context of the invention can be adjusted using acidifying or basifying agents, which are generally present in the composition in proportions preferably of between 0.01% and 30% by weight relative to the total weight of the stripping composition.

Among the acidifying agents that may be mentioned, for example, are mineral or organic acids, for instance hydrochloric acid, orthophosphoric acid, sulfuric acid, carboxylic acids, for instance acetic acid, tartaric acid, citric acid or lactic acid, and sulfonic acids.

Among the basifying agents that may be mentioned, for example, are aqueous ammonia, alkaline silicates, alkaline carbonates, alkanolamines, such as monoethanolamine, diethanolamine and triethanolamine, 2-methyl-2-amino-1-propanol and derivatives thereof, sodium hydroxide, potassium hydroxide and the compounds of formula (II) below: 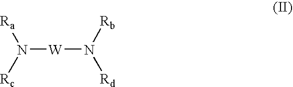
in which W represents a propylene residue optionally substituted with a hydroxyl group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

The stripping composition of the invention may be in any of various forms, such as in the form of solutions, emulsions, creams or gels, optionally pressurized in the form of mousses, or in any other form that is suitable for stripping artificial color from keratin fibers, and in particular from human keratin fibers such as the hair. A preferred embodiment of the invention is hair having the invention composition on at least part of a surface thereof, preferably hair that has been artificially colored, for example dyed with oxidation dyes and/or direct dyes.

A subject of the present invention is also a process for stripping artificial color from keratin fibers, wherein a composition as defined above is applied to the keratin fibers for an action time that is sufficient to strip the artificial color from the keratin fibers.

The application temperature of the stripping composition is not limited, but may generally between 20 and 250° C. and preferably between 20 and 80° C.

The action time that is sufficient to strip the artificial color from the keratin fibers similarly is not limited but can be generally between 1 minute and 120 minutes and preferably between 5 minutes and 60 minutes.

The application conditions, such as the application temperature and the action time, the pH of the stripping composition and the amount of phosphines present in the stripping composition, depend on the amount of artificial dyes to be removed and on the nature of the phosphine(s) used. Those of ordinary skill are capable of such adjustments in view of this disclosure without undue hardship.

A subject of the present invention is also a multi-compartment device for dyeing and then stripping the artificial color from keratin fibers, wherein it comprises a first compartment containing a composition comprising at least one dye precursor and/or a dye, and a second compartment containing a stripping composition as defined above.

According to one particular embodiment of the invention, the composition for dyeing keratin fibers is an oxidation dye composition comprising at least one oxidation base and/or at least one coupler.

The oxidation bases may be chosen from oxidation bases conventionally used in oxidation dyeing. By way of example, these oxidation bases may be chosen from para-phenylenediamines, double bases, para-aminophenols, ortho-aminophenols and heterocyclic bases, and the addition salts thereof.

Among the para-phenylenediamines, mention may be made of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylene-diamine, N-phenyl-para-phenylenediamine, 2-β-hydroxy-ethyloxy-para-phenylenediamine, 2-β-acetylamino-ethyloxy-para-phenylenediamine, N-(β-methoxyethyl)-para-phenylenediamine and 2-methyl-1-N-β-hydroxyethyl-para-phenylenediamine, and the acid-addition salts thereof.

Among the double bases, mention may be made of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-amino-phenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylene-diamine and 1,8-bis(2,5-diaminophenoxy)-3,5-dioxa-octane, and the acid-addition salts thereof.

Among the para-aminophenols, mention may be made of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethyl-phenol and 4-amino-2-(β-hydroxyethylaminomethyl)phenol, and the acid-addition salts thereof.

Among the ortho-aminophenols, mention may be made of 2-aminophenol, 2-amino-1-hydroxy-5-methyl-benzene, 2-amino-1-hydroxy-6-methylbenzene and 5-acetamido-2-aminophenol, and the acid-addition salts thereof.

Among the heterocyclic bases, mention may be made of pyridine derivatives and more particularly the compounds described, for example, in patents GB 1 026 978 and GB 1 153 196, such as 2,5-diamino-pyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the acid-addition salts thereof.

Among the heterocyclic bases, mention may also be made of pyrimidine derivatives and more particularly the compounds described, for example, in German patent DE 2 359 399 or Japanese patents JP 88-169 571 and JP 91-10 659 or patent application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048 and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethyl-pyrazolo[1,5-a]pyrimidine-3,5-diamine; 3-aminopyrazolo-[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)(2-hydroxyethyl)amino]ethanol; 2-[(7-aminopyrazolo[1,5-a]pyrimidin-3-yl)(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine and 3-amino-5-methyl-7-imidazolylpropylamino-pyrazolo[1,5-a]pyrimidine, and the addition salts thereof and the tautomeric forms thereof, when a tautomeric equilibrium exists, and the acid-addition salts thereof.

Among the heterocyclic bases, mention may also be made of pyrazole derivatives, such as the compounds described in patents DE 3 843 892, DE 4 133 957 and patent applications WO 94/08969, WO 94/08970, FR-A-2 733 749 and DE 195 43 988, for example 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methyl-pyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole and 4,5-diamino-1-(β-methoxyethyl)pyrazole, and the acid-addition salts thereof.

The couplers may be chosen from the couplers conventionally used in oxidation dyeing. By way of example, these couplers may be chosen from meta-phenylenediamines, meta-aminophenols, meta-diphenols, naphthalene-based couplers and heterocyclic couplers, and the addition salts thereof.

Among the couplers that may especially be mentioned are 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxy-benzene, 1,3-diamino-benzene, 1,3-bis(2,4-diamino-phenoxy)propane, sesamol, 1-amino-2-methoxy-4,5-methylenedioxybenzene, α-naphthol, 1-acetoxy-2-methylnaphthalene, 2-methyl-1-naphthol, 6-hydroxyindole, 4-hydroxyindole, 4-hydroxy-N-methylindole, 6-hydroxyindoline, 2,6-dihydroxy-4-methylpyridine, 1H-3-methylpyrazol-5-one, 1-phenyl-3-methylpyrazol-5-one, 2-amino-3-hydroxypyridine, 3,6-dimethylpyrazolo-[3,2-c]-1,2,4-triazole and 2,6-dimethylpyrazolo[1,5-b]-1,2,4-triazole, and the acid-addition salts thereof.

In general, the addition salts of the oxidation bases and couplers that may be used in the context of the invention are chosen especially from acid-addition salts such as the hydrochlorides, hydrobromides, sulfates, citrates, succinates, tartrates, lactates, tosylates, benzenesulfonates, phosphates and acetates, and the salts of addition with a base such as sodium hydroxide, potassium hydroxide, ammonia, amines or alkanolamines.

The oxidation base(s) is (are) generally present in the oxidation dye composition in an amount of between 0.0005% and 12% by weight approximately and preferably between 0.005% and 8% by weight approximately relative to the total weight of the dye composition.

The coupler(s) is (are) generally present in the oxidation dye composition in an amount of between 0.0001% and 15% by weight approximately and preferably between 0.001% and 10% by weight approximately relative to the total weight of the dye composition.

According to another particular embodiment of the invention, the composition for dyeing keratin fibers is a direct dye composition comprising one or more direct dyes that may be chosen especially from nitrobenzene dyes, azo direct dyes, methine direct dyes, quinone direct dyes, azine direct dyes, triarylmethane direct dyes, indoamine direct dyes and natural direct dyes. These direct dyes may be of nonionic, anionic or cationic nature.

Among the benzenic direct dyes, mention may be made of 1,4-diamino-2-nitrobenzene, 1-amino-2-nitro-4-(β-hydroxyethylamino)benzene, 1-amino-2-nitro-4-bis(β-hydroxyethyl)aminobenzene, 1,4-bis(β-hydroxyethylamino)-2-nitrobenzene, 1-β-hydroxyethylamino-2-nitro-4-bis(β-hydroxyethylamino)benzene, 1-β-hydroxyethylamino-2-nitro-4-aminobenzene, 1-β-hydroxyethylamino-2-nitro-4-(ethyl)(β-hydroxyethyl)aminobenzene, 1-amino-3-methyl-4-β-hydroxyethylamino-6-nitrobenzene, 1-amino-2-nitro-4-β-hydroxyethylamino-5-chlorobenzene, 1,2-diamino-4-nitrobenzene, 1-amino-2-β-hydroxyethylamino-5-nitrobenzene, 1,2-bis(β-hydroxyethylamino)-4-nitrobenzene, 1-amino-2-[tris(hydroxymethyl)methylamino]-5-nitrobenzene, 1-hydroxy-2-amino-5-nitrobenzene, 1-hydroxy-2-amino-4-nitrobenzene, 1-hydroxy-3-nitro-4-aminobenzene, 1-hydroxy-2-amino-4,6-dinitrobenzene, 1-β-hydroxyethyloxy-2-β-hydroxyethylamino-5-nitrobenzene, 1-methoxy-2-β-hydroxyethylamino-5-nitrobenzene, 1-β-hydroxyethyloxy-3-methylamino-4-nitrobenzene, 1-β,γ-dihydroxypropyloxy-3-methylamino-4-nitrobenzene, 1-p-hydroxyethylamino-4-β,γ-dihydroxypropyloxy-2-nitrobenzene, 1-β,γ-dihydroxypropylamino-4-trifluoromethyl-2-nitrobenzene, 1-β-hydroxyethylamino-4-trifluoromethyl-2-nitrobenzene, 1-β-hydroxyethylamino-3-methyl-2-nitrobenzene, 1-β-aminoethylamino-5-methoxy-2-nitrobenzene, 1-hydroxy-2-chloro-6-ethylamino-4-nitrobenzene, 1-hydroxy-2-chloro-6-amino-4-nitrobenzene, 1-hydroxy-6-[bis(β-hydroxyethyl)amino]-3-nitrobenzene, 1-β-hydroxyethylamino-2-nitrobenzene and 1-hydroxy-4-β-hydroxyethylamino-3-nitrobenzene.

Among the azo direct dyes that may be mentioned are the cationic azo dyes described in patent applications WO 95/15144, WO 95/01772 and EP 0 714 954, the content of which forms an integral part of the invention.

Among these compounds, mention may be made most particularly of 1,3-dimethyl-2-[[4-(dimethylamino)phenyl]azo]-1H-imidazolium chloride, 1,3-dimethyl-2-[(4-aminophenyl)azo]-1H-imidazolium chloride and 1-methyl-4-[(methylphenylhydrazono)methyl]-pyridinium methyl sulfate.

Among the azo direct dyes that may also be mentioned are the following dyes described in the Color Index International 3rd edition: Disperse Red 17; Acid Yellow 9; Acid Black 1; Basic Red 22; Basic Red 76; Basic Yellow 57; Basic Brown 16; Acid Yellow 36; Acid Orange 7; Acid Red 33; Acid Red 35; Basic Brown 17; Acid Yellow 23; Acid Orange 24; Disperse Black 9.

Mention may also be made of 1-(4′-aminodiphenylazo)-2-methyl-4-[bis(β-hydroxyethyl)amino]benzene and 4-hydroxy-3-(2-methoxyphenylazo)-1-naphthalene-sulfonic acid.

Among the quinone direct dyes that may be mentioned are the following dyes: Disperse Red 15; Solvent Violet 13; Acid Violet 43; Disperse Violet 1; Disperse Violet 4; Disperse Blue 1; Disperse Violet 8; Disperse Blue 3; Disperse Red 11; Acid Blue 62; Disperse Blue 7; Basic Blue 22; Disperse Violet 15; Basic Blue 99, and also the following compounds: 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone and 1,4-bis(β,γ-dihydroxypropylamino)anthraquinone.

Among the azine dyes that may be mentioned are the following compounds: Basic Blue 17 and Basic Red 2.

Among the triarylmethane dyes, mention may be made of the following compounds: Basic Green 1; Acid Blue 9; Basic Violet 3; Basic Violet 14; Basic Blue 7; Acid Violet 49; Basic Blue 26; Acid Blue 7.

Among the indoamine dyes, mention may be made of the following compounds: 2-β-hydroxyethylamino-5-[bis(β-4′-hydroxyethyl)amino]anilino-1,4-benzoquinone, 2-β-hydroxyethylamino-5-(2′-methoxy-4¹-amino)anilino-1,4-benzoquinone, 3-N(2′-chloro-4′-hydroxy)phenylacetylamino-6-methoxy-1,4-benzoquinoneimine, 3-N(3′-chloro-4′-methylamino)phenylureido-6-methyl-1,4-benzoquinoneimine and 3-[4′-N-(ethylcarbamylmethyl)amino]phenylureido-6-methyl-1,4-benzoquinoneimine.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin and apigenidin. Extracts or decoctions containing these natural dyes may also be used, and especially henna-based poultices or extracts.

The direct dye(s) is (are) generally present in an amount of between 0.001% and 20% by weight approximately and even more preferably between 0.005% and 10% by weight approximately relative to the total weight of the dye composition.

According to another particular embodiment of the invention, the dye composition is a composition comprising at least one oxidation base, optionally at least one coupler, and at least one direct dye.

The multi-compartment device in accordance with the invention may also comprise a third compartment containing an oxidizing composition.

The examples that follow serve to illustrate the invention without, however, being limiting in nature.

### EXAMPLE 1

A lock of 1 g of natural hair containing 90% white hairs was predyed with a dye Majirouge 6,66 under the following conditions: 5 g of dye mixture, prepared in proportions of 1+1.5 between the dye support and the 20-volumes oxidizing agent sold by L'Oréal Professionel, were applied to the lock for an action time of 35 minutes at 33° C. The lock was then rinsed, washed with a DOP shampoo and then dried.

The lock was then stripped by immersion for 30 minutes in the composition described in the table below, at a temperature of 33° C., at a rate of 10 g of composition per 1 g of hair. The lock was then rinsed, and then shampooed and dried.

| | Stripping composition |
|---|---|
| tri(hydroxymethyl)phosphine | 5 g |
| sodium lauryl sulfate | 3 g |
| hydroxypropyl guar | 1 g |
| phosphoric acid | qs pH = 3 |
| demineralized water | qs 100 g |

To check that the stripping of the artificial dyes from the keratin fibers is efficient, an aqueous 20-volumes hydrogen peroxide solution was applied to the keratin fibers for 3 minutes. The lock was then rinsed, and then shampooed with a DOP shampoo and dried.

This treatment did not produce any perceptible change in the color. The composition comprising the phosphine did indeed make it possible to remove the dyes present in a single step.

The calorimetric values of the dried lock were taken just before stripping and after stripping, using a Minolta CM-2022 spectrocolorimeter. The color difference thus calculated (ΔE) is 30, which corresponds for this type of shade to substantial stripping. The slight residual color is not in any way harmful as regards recoloration.

### EXAMPLE 2

A lock of 1 g of natural hair containing 90% white hairs was predyed with a dye Majimix 0,600 under the following conditions: 5 g of dye mixture, prepared in proportions of 1+1.5 between the dye support and the 20-volumes oxidizing agent sold by L'Oréal Professionel, were applied to the lock for an action time of 35 minutes at 33° C. The lock was then rinsed, washed with a DOP shampoo and then dried.

The lock was then stripped by immersion for 30 minutes in the composition described in the table below, at a temperature of 33° C., at a rate of 10 g of composition per 1 g of hair. The lock was then rinsed, and then shampooed and dried.

| | Stripping composition |
|---|---|
| Tri(hydroxymethyl)phosphine | 5 g |
| sodium lauryl sulfate | 3 g |
| hydroxypropyl guar | 1 g |
| phosphoric acid | qs pH = 3 |
| demineralized water | qs 100 g |

To check that the stripping of the artificial dyes from the keratin fibers is efficient, an aqueous 20-volumes hydrogen peroxide solution was applied to the keratin fibers for 3 minutes. The lock was rinsed and then shampooed with a DOP shampoo and dried.

This treatment did not induce any perceptible change in the color. The composition comprising the phosphine did indeed make it possible to remove the dyes present in a single step.

The colorimetric values of the dried lock were taken just before stripping and after stripping, using a Minolta CM-2022 spectrocolorimeter. The color difference thus calculated (ΔE) is 29.3, which corresponds for this type of shade to substantial stripping. The slight residual color is not in any way harmful as regards recoloration.

### EXAMPLE 3

A lock of 1 g of natural hair containing 90% white hairs was predyed with a commercial dye Movida 45 under the following conditions: 5 g of dye mixture, prepared according to the conditions recommended in the kit, were applied to the lock for an action time of 15 minutes at 33° C. The lock was then rinsed, washed with a DOP shampoo and then dried.

The lock was then stripped by immersion for 30 minutes in the composition described in the table below, at a temperature of 33° C., at a rate of 10 g of composition per 1 g of hair. The lock was then rinsed, and then shampooed and dried.

| | Stripping composition |
|---|---|
| tri(hydroxymethyl)phosphine | 5 g |
| sodium lauryl sulfate | 3 g |
| hydroxypropyl guar | 1 g |
| phosphoric acid | qs pH = 3 |
| demineralized water | qs 100 g |

To check that the stripping of the artificial dyes from the keratin fibers is efficient, an aqueous 20-volumes hydrogen peroxide solution was applied to the keratin fibers for 3 minutes. The lock was then rinsed, and then shampooed with a DOP shampoo and dried.

This treatment did not induce any perceptible change in the color. The composition comprising the phosphine did indeed make it possible to remove the dyes present in a single step.

The calorimetric values of the dried lock were taken just before stripping and after stripping, using a Minolta CM-2022 spectrocolorimeter. The color difference thus calculated (ΔE) is 4.8, which corresponds for this type of shade to appreciable stripping. The residual color is not harmful as regards recoloration.

The above written description of the invention provides a manner and process of making and using it such that any person skilled in this art is enabled to make and use the same, this enablement being provided in particular for the subject matter of the appended claims, which make up a part of the original description and including the use, for stripping artificial color from keratin fibers, of a composition comprising, in a cosmetically acceptable medium, at least one phosphine or an acid-addition salt thereof as major reducing agent. Also fully described and enabled herein is a process for stripping artificial color from keratin fibers, wherein a composition as defined herein is applied to the keratin fibers for an action time that is sufficient to strip the artificial color from the keratin fibers, and a multi-compartment device for dyeing and then stripping artificial color from keratin fibers, wherein it comprises a first compartment containing a composition comprising at least one dye precursor and/or a dye, and a second compartment containing a stripping composition as defined herein.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for stripping artificial color from keratin fibers, comprising contacting keratin fibers in need thereof with a composition comprising, in a cosmetically acceptable medium, at least one phosphine and/or an acid-addition salt thereof, wherein when said composition comprises one or more reducing agents other than said at least one phosphine and/or acid-addition salt thereof, said reducing agent(s) are present in a weight amount less than the weight amount of the at least one phosphine and/or an acid-addition salt thereof.

2. The method according to claim 1, in which the at least one phosphine and/or an acid-addition salt thereof is (are) chosen from compounds of formula (I) and acid-addition salts thereof: 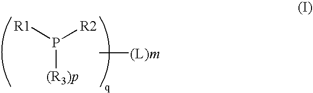
in which:
L is a linker that represents a covalent bond or a divalent hydrocarbon-based radical optionally comprising one or more hetero atoms chosen from an oxygen atom, a sulfur atom, a nitrogen atom and a silicon atom;
m is an integer equal to 0 or 1;
q is an integer equal to 1 or 2;
p is an integer equal to 0 or 1;
R₁, R₂ and R₃, which may be identical or different, represent:
a hydrogen atom;
a halogen atom;
a hydroxyl radical;
a carboxyl radical;
a monovalent hydrocarbon-based radical optionally comprising one or more hetero atoms chosen from a sulfur atom, an oxygen atom, a nitrogen atom, a phosphorus atom and a silicon atom, optionally substituted with one or more radicals chosen from:
a halogen atom,
a hydroxyl radical,
an alkoxy radical,
a haloalkyl radical,
an amino radical,
a carboxyl radical,
an alkoxycarbonyl radical,
an amido radical,
an alkylaminocarbonyl radical,
an acylamino radical,
a mono- or di(alkyl)amino radical,
a mono- or di(hydroxyalkyl)amino radical,
an N-aryl-N-alkylamino radical,
an aromatic or heteroaromatic ring, which is unsubstituted or substituted with one or more radicals chosen from a halogen atom, a hydroxyl radical, an alkoxy radical and a mono- or di(alkyl)amino radical,
a cyano radical,
a radical that increases the solubility of the phosphine in water,
a substituted or unsubstituted, aromatic or non-aromatic heterocyclic radical;
a substituted or unsubstituted aryl radical;
a substituted or unsubstituted arylalkyl radical;
an arylalkyloxy radical;
a substituted or unsubstituted, aromatic or non-aromatic heterocyclic radical;
a silyl radical;
wherein:
when q=1, m=0 and p=1;
when q=2, m=1 and p=0 or 1, with:
when p=0, the linker L is attached to the phosphorus atom;
when p=1, the linker L is attached to one of the radicals R₁, R₂ and R₃.

3. The method according to claim 2, in which the radicals R₁, R₂ and R₃ do not simultaneously represent a hydrogen atom.

4. The method according to claim 2, in which at least one of the radicals R₁, R₂ and R₃ represents a hydrocarbon-based radical chosen from optionally substituted alkyl radicals.

5. The method according to claim 2, in which the radicals R₁, R₂ and R₃ are chosen from a hydrogen atom; an alkyl radical; a cycloalkyl radical optionally substituted with one or more alkyl radicals; an alkoxy radical; an alkoxyalkyl radical; a haloalkyl radical; a cyanoalkyl radical; a hydroxyalkyl radical; a carboxyalkyl radical; a halogen atom; a hydroxyl radical; a carboxyl radical; an alkenyl radical; a mono- or dialkylamino radical; an N-aryl-N-alkylaminoalkyl radical; an aryl radical optionally substituted with one or more radicals chosen from an alkyl radical, an alkoxy radical, a mono- or dialkylamino radical, a mono- or dialkylaminoalkyl radical, a haloalkyl radical, a hydroxyl radical, a carboxyl radical, a halogen atom, an aryl radical substituted with a mono- or dialkylaminoalkyl radical; an arylalkyl radical; an arylalkyloxy radical; a pyrrolidino radical; a furyl radical; a morpholino radical; a thienyl radical; a pyridyl radical; a trialkylsilyl radical; an alkyl radical substituted with a pyrrolidino radical, a furyl radical, a morpholino radical or a thienyl radical.

6. The method according to claim 5, in which the radicals R₁, R₂ and R₃, which may be identical or different, are chosen from a hydrogen atom; a methyl radical; an ethyl radical; a propyl radical; an isopropyl radical; an n-butyl radical; an isobutyl radical; a tert-butyl radical; an octyl radical; a cyclohexyl radical; a cyclopentyl radical; a methoxy radical; an ethoxy radical; a methoxypropyl radical; a chloroethyl radical; a cyanoethyl radical; a hydroxymethyl radical; a hydroxypropyl radical; a carboxyethyl radical; a chlorine atom; a hydroxyl radical; a carboxyl radical; a trifluoromethyl radical; a chloromethyl radical; an allyl radical; a vinyl radical; a dimethylamino radical; a diethylamino radical; a di(isopropyl)amino radical; a phenyl radical; an o-tolyl radical; an m-tolyl radical; a p-tolyl radical; a dimethylphenyl radical; a trimethylphenyl radical; an o-methoxyphenyl radical; an m-methoxyphenyl radical; a p-methoxyphenyl radical; a dimethoxyphenyl radical; a trimethoxyphenyl radical; an o-(dimethylamino)phenyl radical; an m-(dimethylamino)phenyl radical; a p-(dimethylamino)phenyl radical; a di(tert-butyl)phenyl radical; a tri(tert-butyl)phenyl radical; a trifluoromethylphenyl radical; a bis(tri-fluoromethyl)phenyl radical; an o-fluorophenyl radical; an m-fluorophenyl radical; a p-fluorophenyl radical; an o-chlorophenyl radical; an m-chlorophenyl radical; a p-chlorophenyl radical; an o-hydroxyphenyl radical; an m-hydroxyphenyl radical; a p-hydroxyphenyl radical; a 4-(diethylaminomethyl)phenyl radical; a 3,5-dimethyl-4-methoxyphenyl radical; a 2-methylbiphenyl radical; a benzyl radical; a benzyloxy radical; a naphthyl radical; a morpholino radical; a morpholinomethyl radical; a pyrrolidino radical; a furyl radical; a pyridyl radical; a thienyl radical; a trimethylsilyl radical; a 2-(4-diethylaminomethylphenyl)phenyl radical; a 5-methyl-2-isopropylcyclohexyl radical; an N-methyl-N-phenylaminomethyl radical; a carboxyphenyl radical.

7. The method according to claim 1, in which the at least one phosphine and/or an acid-addition salt thereof is (are) chosen from monophosphines and acid-addition salts thereof.

8. The method according to claim 2, in which in formula (I) q is equal to 1.

9. The method according to claim 1, wherein said composition comprises at least one of the following materials and/or an addition salt thereof: tri(hydroxymethyl)phosphine; tri(hydroxypropyl)phosphine; bis(hydroxymethyl)(phenyl)phosphine; allyldiphenylphosphine; benzyldiphenylphosphine; bis(3,4,5-trimethoxyphenyl)chlorophosphine; bis(3,4,5-trimethoxyphenyl)phosphine; benzyloxy(diisopropylamino)methylphosphine; bis(diisopropylamino)chlorophosphine; bis(2-cyanoethyl)phosphine; bis(3,5-di-tert-butylphenyl)chlorophosphine; bis(3,5-di-tert-butyl-phenyl)phosphine; bis(diethylamino)methylphosphine; bis(diethylamino)chlorophosphine; bis(diethylamino)phenylphosphine; bis(3,5-dimethyl-4-methoxyphenyl)chlorophosphine; bis(3,5-dimethyl-4-methoxyphenyl)phosphine; bis(3,5-dimethylphenyl)chlorophosphine; bis(3,5-dimethylphenyl)diethylaminophosphine; bis(3,5-dimethylphenyl)phosphine; bis(3,5-ditrifluoromethylphenyl)chlorophosphine; bis(3,5-ditrifluoromethylphenyl)phosphine; bis(4-fluorophenyl)chlorophosphine; bis(2-furyl)chlorophosphine; bis(2-furyl)phosphine; bis(hydroxymethyl)phenylphosphine; bis(4-methoxyphenyl)phenylphosphine; bis(3,5-dimethylphenyl)phosphine; bis(3,5-di-tert-butylphenyl)chlorophosphine; bis(3,5-di-tert-butylphenyl)phosphine; bis(3,5-ditrifluoromethylphenyl)chlorophosphine; bis(3,5-ditrifluoromethylphenyl)phosphine; bis(4-fluorophenyl)chlorophosphine; bis(4-methoxyphenyl)chlorophosphine; bis(4-methoxyphenyl)phenylphosphine; bis(4-methylphenyl)chlorophosphine; bis(4-methylphenyl)phosphine; bis(4-trifluoromethylphenyl)chlorophosphine; bis(4-trifluoromethylphenyl)phosphine; bis(diethylamino)methylphosphine; bis(diethylamino)phenylphosphine; bis(hydroxymethyl)phenylphosphine; bis(o-tolyl)chlorophosphine; bis(o-tolyl)phosphine; bis(pyrrolidino)methylphosphine; butyldichlorophosphine; butyldiphenylphosphine; tert-butyldiphenylphosphine; cyclohexyl(diethylamino)chlorophosphine; cyclohexyl(dimethyl-amino)chlorophosphine; cyclohexyldichlorophosphine; cyclohexyldiphenylphosphine; 2-chloroethyldiphenylphosphine; 2-(dicyclohexylphosphino)biphenyl; 2-dicyclo-hexylphosphino-2′-(N,N-dimethylamino)biphenyl; diethyl-aminodiethylphosphine; dimethylaminodichlorophosphine; (4-dimethylaminophenyl)diphenylphosphine; N-[(diphenyl-phosphinyl)methyl]-N-methylaniline; o-diphenylphosphinobenzoic acid; 2-methoxy(dichlorophosphino)benzene; 4-methoxyphenyl(diethylamino)chlorophosphine; 4-methoxyphenyl(dimethylamino)chlorophosphine; (2-methoxyphenyl)methylphenylphosphine; 2-methoxyphosphinobenzene; (5-methyl-2-isopropylcyclohexyl)diphenyl-phosphine; triphenylphosphine; diallylphenylphosphine; dibenzylphosphine; dibutylphenylphosphine; dibutyl-phosphine; dicyclohexylchlorophosphine; dicyclohexyl-phenylphosphine; dicyclohexylphosphine; diethylchloro-phosphine; diethylphenylphosphine; diethylphosphine; diisobutylphosphine; diisopropylchlorophosphine; diisopropylphosphine; dimethyl(phenyl)phosphine; dimethyl(trimethylsilyl)phosphine; dimethylchlorophosphine; diphenyl(o-tolyl)phosphine; diphenyl(p-tolyl)phosphine; diphenyl(trimethylsilyl)phosphine; diphenyl-chlorophosphine; diphenylphosphine; diphenylpropyl-phosphine; diphenylvinylphosphine; di-tert-butylchlorophosphine; di-tert-butylhydroxyphosphine; di-tert-butylmethylphosphine; di-tert-butylphenylphosphine; di-tert-butylphosphine; divinylphenylphosphine; ethyl-dichlorophosphine; ethyldiphenylphosphine; isopropyl-dichlorophosphine; methoxydiethoxyphosphine; methyl-dichlorophosphine; methyldiphenylphosphine; methyl-phenylchlorophosphine; phenylphosphine; propyldichlorophosphine; tert-butylbis(trimethylsilyl)phosphine; tert-butyldichlorophosphine; tert-butyldiethylphosphine; tert-butyldiphenylphosphine; tert-butylphosphine; tri(m-tolyl)phosphine; tri(o-tolyl)phosphine; tri(p-tolyl)phosphine; tricyclohexylphosphine; tricyclopentylphosphine; triethylphosphine; triisobutylphosphine; triisopropylphosphine; trimethylphosphine; tri-n-butylphosphine; tri-n-octylphosphine; tripropylphosphine; tris(1-naphthyl)phosphine; tris(2,4,6-trimethylphenyl)phosphine; tris(2,6-dimethoxyphenyl)phosphine; tris(2-carboxyethyl)phosphine; tris(2-cyanoethyl)phosphine; tris(2-furyl)phosphine; tris(2-methoxyphenyl)phosphine; tris(2-thienyl)phosphine; tris(3,5-dimethyl-4-methoxy)phosphine; tris(3-chlorophenyl)phosphine; tris(3-fluoro-phenyl)phosphine; tris(3-methoxyphenyl)phosphine; tris(3-methoxypropyl)phosphine; tris(4-chlorophenyl)phosphine; tris(4-fluorophenyl)phosphine; tris(4-methoxyphenyl)phosphine; tris(4-morpholino)phosphine; tris(hydroxymethyl)phosphine; tris(trimethylsilyl)phosphine; tris[3,5-bis(trifluoromethyl)phenyl]phosphine; tri-tert-butylphosphine; 2-cyanoethyldiphenylphosphine; 2-dicyclohexylphosphino-2′-methylbiphenyl; bis(2,4,6-trimethylphenyl)phosphine; 2-(di-tert-butyl-phosphino)biphenyl.

10. The method according to claim 9, wherein said composition comprises at least one of the following materials and/or an addition salt thereof:
trihydroxymethylphosphine; trihydroxypropylphosphine; bis(hydroxymethyl)phenylphosphine.

11. The method according to claim 1, wherein said composition comprises at least one diphosphine and/or an addition salt thereof.

12. The method according to claim 2, in which q is equal to 2.

13. The method according to claim 12, in which p is equal to 0 and the linker L is a covalent bond or a divalent radical chosen from a binaphthylene radical; a methylene radical; an ethylene radical; a propylene radical; a butylene radical; a pentylene radical; a hexylene radical; a phenylene radical; a meta-dimethylenebenzene radical; an N-methyl-N′-methylhydrazo radical; a vinylene radical; a diethyleneoxy radical.

14. The method according to claim 1, wherein said composition comprises at least one of the following materials and/or an acid addition salt thereof: 2,2′-bis(dicyclohexylphosphino)-1,1′-binaphthyl; 2,2′-bis[bis(3,5-dimethylphenylphosphino)]-1,1′-binaphthyl; 1,4-bis[bis(3,5-dimethylphenyl)phosphino]butane; 1,2-bis[bis(3,5-dimethylphenyl)phosphino]ethane; bis[bis(3,5-dimethylphenyl)phosphino]methane; 1,5-bis[bis(3,5-dimethylphenyl)phosphino]-pentane; 1,3-bis[bis(3,5-dimethylphenyl)phosphino]-propane; 2,2′-bis[bis(3,5-ditrifluoromethylphenyl)phosphino]-1,1′-binaphthyl; 1,4-bis[bis(3,5-ditrifluoromethylphenyl)phosphino]butane; 1,2-bis[bis(3,5-ditrifluoromethylphenyl)phosphino]ethane; bis[bis(3,5-ditrifluoromethylphenyl)phosphino]methane; 1,5-bis-[bis(3,5-ditrifluoromethylphenyl)phosphino]pentane; 1,3-bis[bis(3,5-ditrifluoromethylphenyl)phosphino]-propane; 1,2-bis(di-tert-butylphosphino)benzene; 1,4-bis(di-tert-butylphosphino)butane; 1,2-bis(di-tert-butylphosphino)ethane; 1,3-bis(di-tert-butylphosphino-methyl)benzene; 1,3-bis(di-tert-butylphosphino)propane; 1,2-bis(dichlorophosphino)benzene; 1,3-bis(dichloro-phosphino)benzene; 1,4-bis(dichlorophosphino)benzene; 1,4-bis(dichlorophosphino)butane; 1,2-bis(dichloro-phosphino)-1,2-dimethylhydrazine; 1,2-bis(dichloro-phosphino)ethane; bis(dichlorophosphino)methane; 1,3-bis(dichlorophosphino)propane; 1,2-bis(dicyclohexyl-phosphino)benzene; 2,2′-bis(dicyclohexylphosphino)-1,1′-binaphthyl; 1,4-bis(dicyclohexylphosphino)butane; (2R,3R)bis(dicyclohexylphosphino)butane; (2S,3S)-bis(dicyclohexylphosphino)butane; 1,2-bis(dicyclohexyl-phosphino)ethane; bis(dicyclohexylphosphino)methane; 1,3-bis(dicyclohexylphosphino)propane; bis[2-(4-diethylaminomethylphenyl)phenylphosphino]ethyl ether; 1,2-bis(diethylphosphino)ethane; 1,2-bis(dimethyl-phosphino)benzene; 1,4-bis(dimethylphosphino)butane; 1,2-bis(dimethylphosphino)ethane; bis(dimethylphosphino)methane; 1,3-bis(dimethylphosphino)propane; 1,2-bis(diphenylphosphino)benzene; 1,3-bis(diphenylphosphino)benzene; 1,4-bis(diphenylphosphino)benzene; 2,2′-bis(diphenylphosphino)-1,1′-binaphthyl; 1,4-bis(di-phenylphosphino)butane; 1,2-bis(diphenylphosphino)ethane; cis-1,2-bis(diphenylphosphino)ethylene; trans-1,2-bis(diphenylphosphino)ethylene; bis(2-diphenylphosphino)ethyl ether; 1,6-bis(diphenylphosphino)hexane; bis(diphenylphosphino)methane; 1,5-bis(diphenylphosphino)pentane; 1,3-bis(diphenylphosphino)propane; 1,2-bis(ditrifluoromethylphosphino)ethane; 1,2-bis[(2-methoxyphenyl)phenylphosphino]ethane; 1,2-bis-(phenylphosphino)ethane; 1,3-bis(phenylphosphino)propane; bis-2-[(phenyl)(3-pyridyl)phosphinoethyl) ether; 1,2-bis(phosphino)benzene; 1,2-bis(phosphino)ethane; bis(phosphino)methane; 1,2-bis(trifluoro-methyl)phosphino)ethane; bis(di-tert-butylphosphino)pentane; tetraphenylbiphosphine.

15. The method according to claim 1, wherein the at least one phosphine and/or an acid-addition salt thereof is (are) compounds that are soluble in the cosmetically acceptable medium.

16. The method according to claim 15, in which the at least one phosphine and/or an acid-addition salt thereof is (are) water-soluble.

17. The method according to claim 1, wherein the at least one phosphine and/or an acid-addition salt thereof is the sole reducing agent in said composition.

18. The method according to claim 1, wherein the at least one phosphine and/or an acid-addition salt thereof is (are) present in an amount of between 0.001% and 30% by weight relative to the total weight of the composition.

19. The method according to claim 1, wherein the pH of the composition is between 2 and 12.

20. The method according to claim 19, wherein said composition comprises trihydroxymethylphosphine and the pH of the composition is about 3.

21. The method according to claim 19, wherein said composition comprises trihydroxypropylphosphine and the pH of the composition is about 9.

22. The method according to claim 1, wherein said composition is applied to the keratin fibers for an action time that is sufficient to strip the artificial color from the keratin fibers.

23. A multi-compartment device for dyeing and stripping artificial color from keratin fibers, comprising a first compartment comprising a composition, said composition comprising at least one dye precursor and/or a dye, and a second compartment comprising a composition comprising, in a cosmetically acceptable medium, at least one phosphine and/or an acid-addition salt thereof, wherein when said composition comprises one or more reducing agents other than said at least one phosphine and/or an acid-addition salt thereof, said reducing agents are present in a weight amount less than the weight amount of the at least one phosphine or an acid-addition salt thereof.

24. The device according to claim 23, in which the dye precursor(s) is (are) chosen from oxidation bases and/or couplers.

25. The device according to claim 23, in which the dye(s) is (are) chosen from direct dyes.

26. The device according to claim 23, further comprising a third compartment comprising an oxidizing composition.
